(19) United States
(12) Patent Application Publication
(10) Pub. No.: US 2015/0164874 A1
(43) Pub. Date: Jun. 18, 2015

(54) PIRFENIDONE AND ANTI-FIBROTIC THERAPY IN SELECTED PATIENTS

(71) Applicant: Williamson Z. Bradford, Wilson, WY (US)

(72) Inventor: Williamson Z. Bradford, Wilson, WY (US)

(73) Assignee: INTERMUNE, INC., Brisbane, CA (US)

(21) Appl. No.: 14/373,276

(22) PCT Filed: May 25, 2012
(86) PCT No.: PCT/US12/39538

§ 371 Date: Feb. 26, 2015

Related U.S. Application Data
(60) Provisional application No. 61/489,936, filed on May 25, 2011.
(60) Provisional application No. 61/490,057, filed on May 26, 2011.
(60) Provisional application No. 61/523,047, filed on Aug. 12, 2011.
(60) Provisional application No. 61/524,961, filed on Aug. 18, 2011.

(51) Int. Cl. A61K 31/4418; A61K 38/21

(52) U.S. Cl. CPC A61K 31/4418; A61K 38/217

(57) ABSTRACT

The present invention relates to methods of treating pulmonary fibrosis with pirfenidone and/or other agents.

### CROSS-REFERENCE TO RELATED APPLICATION

The benefit of U.S. Provisional Application No. 61/489,936, filed May 25, 2011; Application No. 61/490,057, filed May 26, 2011; Application No. 61/523,047, filed Aug. 12, 2011; and Application No. 61/524,961, filed Aug. 18, 2011, is claimed, the disclosures of each is incorporated by reference in its entirety.

### FIELD OF THE INVENTION

The invention relates to improved methods of administering pirfenidone and anti-fibrotic therapy involving selected patient populations.

### BACKGROUND

Pirfenidone is small molecule with a molecular weight of 185.23 daltons whose chemical name is 5-methyl-1-phenyl-2-(1H)-pyridone. Pirfenidone has anti-fibrotic properties and has been investigated for therapeutic benefits to patients suffering from various fibrotic conditions. It is approved in Japan for treatment of idiopathic pulmonary fibrosis (IPF) under the trade name Pirespa®. It is approved in Europe for treatment of IPF under the trade name Esbriet®.

### SUMMARY OF THE INVENTION

The invention disclosed herein is based on the identification of a population of patients with idiopathic pulmonary fibrosis (IPF) for whom pirfenidone provides a greater magnitude of relative benefit.

In some aspects, the invention provides a method of administering pirfenidone therapy to a patient with pulmonary fibrosis (e.g., a patient with IPF), wherein said patient is selected, or diagnosed, or identified to have one or more of the following criteria: (1) ratio of forced expiratory volume in one second (FEV1) to forced vital capacity volume (FVC), or FEV1/FVC, is greater than 0.80, (2) percent of predicted FVC (% FVC) is 90% or less, for example ranging from 50% to 90%, inclusive of both endpoints, and (3) time since diagnosis of IPF is at least six months and up to 48 months. The terms “selecting”, “diagnosing” and “identifying” are used synonymously with respect to a patient.

A related aspect of the invention provides pirfenidone for use in treating pulmonary fibrosis in the selected patient population described herein. Similarly, a further related aspect of the invention provides the use of pirfenidone in the manufacture of a medicament for treating pulmonary fibrosis in the selected patient population described herein. It is understood that any of the aspects or embodiments or examples described herein with respect to methods of treatment apply to aspects of the invention that provide pirfenidone for use in treating pulmonary fibrosis, or use of pirfenidone in preparation of a medicament for treating pulmonary fibrosis.

In additional or alternative aspects, the invention provides a method of administering an agent to a patient with pulmonary fibrosis (e.g., a patient with IPF), wherein said patient is selected, or diagnosed, or identified to have one or more of the following criteria: (1) ratio of forced expiratory volume in one second (FEV1) to forced vital capacity volume (FVC), or FEV1/FVC, is greater than 0.80, (2) percent of predicted FVC (% FVC) is 90% or less, for example ranging from 50% to 90%, inclusive of both endpoints, and/or (3) time since diagnosis of IPF is at least six months and up to 48 months, and wherein the agent is selected from steroids (including but not limited to prednisolone), cytotoxic agents (including but not limited to azathioprine and cyclophosphamide), bardoxolone, LPA antagonists (including but not limited to AM152); Torisel (temsirolimus); PI3K inhibitors; pentraxin (including but not limited to Pentraxin-2 (PTX-2 or PRM-151)); MEK inhibitors (including but not limited to ARRY-162 and ARRY-300); p38 inhibitors; PAI-1 inhibitors (including but not limited to Tiplaxtinin); agents that reduce the activity of transforming growth factor-beta (TGF-β) (including but not limited to pan TGF-β neutralizing antibodies, such as GC-1008 (Genzyme/MedImmune); anti-TGF-β2 mAbs, such as lerdelimumab (CAT-152; Trabio, Cambridge Antibody); anti-TGF-β1 antibodies, such as metelimumab (CAT-192,Cambridge Antibody); small molecule TGF-βR1 inhibitors, such as LY-2157299 (Eli Lilly); ACU-HTR-028 (Opko Health)) including antibodies that target one or more TGF-β isoforms, inhibitors of TGF-β receptor kinases TGFBR1 (ALKS) and TGFBR2, and modulators of post-receptor signaling pathways; modulators of chemokine receptor signaling; endothelin receptor antagonists including inhibitors that target both endothelin receptor A and B and those that selectively target endothelin receptor A (including but not limited to ambrisentan; avosentan; bosentan; clazosentan; darusentan; BQ-153; FR-139317, L-744453; macitentan; PD-145065; PD-156252; PD163610;PS-433540; S-0139; sitaxentan sodium; TBC-3711; zibotentan); agents that reduce the activity of connective tissue growth factor (CTGF) (including but not limited to FG-3019, FibroGen), and also including other CTGF-neutralizing antibodies, such as FG-3019; matrix metalloproteinase (MMP) inhibitors (including but not limited to MMPI-12, PUP-1 and tigapotide triflutate, and doxycycline, marimastat, and cipemastat); agents that reduce the activity of epidermal growth /factor receptor (EGFR) including but not limed to erlotinib, gefitinib, BMS-690514, cetuximab, antibodies targeting EGF receptor, inhibitors of EGF receptor kinase, and modulators of post-receptor signaling pathways; agents that reduce the activity of platelet derived growth factor (PDGF) (including but not limited to Imatinib mesylate (Novartis)) and also including PDGF neutralizing antibodies, antibodies targeting PDGF receptor (PDGFR), inhibitors of PDGFR kinase activity, and post-receptor signaling pathways; agents that reduce the activity of vascular endothelial growth factor (VEGF) (including but not limited to axitinib, bevacizumab, BIBF-1120, CDP-791, CT-322, IMC-18F1, PTC-299, and ramucirumab) and also including VEGF-neutralizing antibodies, antibodies targeting the VEGF receptor 1 (VEGFR1, Flt-1) and VEGF receptor 2 (VEGFR2, KDR), the soluble form of VEGFR1 (sFlt) and derivatives thereof which neutralize VEGF, and inhibitors of VEGF receptor kinase activity; inhibitors of multiple receptor kinases such as BIBF-1120 which inhibits receptor kinases for vascular endothelial growth factor, fibroblast growth factor, and platelet derived growth factor; agents that interfere with integrin function (including but not limited to STX-100 and IMGN-388) and also including integrin targeted antibodies; agents that interfere with the pro-fibrotic activities of IL-4 (including but not limited to AER-001, AMG-317, APG-201, and sIL-4Rα) and IL-13 (including but not limited to AER-001, AMG-317, anrukinzumab, CAT-354, cintredekin besudotox, MK-6105, QAX-576, SB-313, SL-102, and TNX-650) and also including neutralizing anti-bodies to either cytokine, antibodies that target IL-4 receptor or IL-13 receptor, the soluble form of IL-4 receptor or derivatives thereof that is reported to bind and neutralize both IL-4 and IL-13, chimeric proteins including all or part of IL-13 and a toxin particularly pseudomonas endotoxin, signaling though the JAK-STAT kinase pathway; agents that interfere with epithelial mesenchymal transition including inhibitors of mTor (including but not limited to AP-23573 or rapamycin); agents that reduce levels of copper such as tetrathiomolybdate; agents that reduce oxidative stress including N-acetyl cysteine and tetrathiomolybdate; and interferon gamma. Also contemplated are agents that are inhibitors of phosphodiesterase 4 (PDE4) (including but not limited to Roflumilast); inhibitors of phosphodiesterase 5 (PDE5) (including but not limited to mirodenafil, PF-4480682, sildenafil citrate, SLx-2101, tadalafil, udenafil, UK-369003, vardenafil, and zaprinast); or modifiers of the arachidonic acid pathway including cyclooxygenase and 5-lipoxegenase inhibitors (including but not limited to Zileuton). Further contemplated are compounds that reduce tissue remodeling or fibrosis including prolyl hydrolase inhibitors (including but not limited to 1016548, CG-0089, FG-2216, FG-4497, FG-5615, FG-6513, fibrostatin A (Takeda), lufironil,P-1894B, and safironil) and peroxisome proliferator-activated receptor (PPAR)-gamma agonists (including but not limited to pioglitazone and rosiglitazone). The method disclosed can comprise administering an agent as disclosed directly above and/or an agent selected from BG-12, chemokine activity modulators (including but not limited to CNTO 888, an antibody targeting CCL2), Lys1 oxidase inhibitors (including but not limited to AB0024/GS-6624, an antibody targeting human lysyl oxidase-like 2), NOX4 inhibitors (including but not limited to GKT137831, a selective NOX 1/4 inhibitor), angiotensin II receptor antagonists (including but not limited to lorsartan), inhibitors or Wnt-beta catenin signaling agents (including but not limited to ICG-001); JNK inhibitors (including but not limited to CC930); IL-4/IL-13 antibody/soluble receptors (including but not limited to SAR156597), and a deuterated pirfenidone (as described e.g., in W009/035598 and having one to fourteen deuterium atoms replacing a hydrogen atom in pirfenidone).

### BRIEF DESCRIPTION OF THE DRAWINGS

FIGS. 1A-1D depicts a analysis of FVC progression from a clinical trial involving a different drug, interferon gamma-lb, which is inert with respect to effect on efficacy outcomes, in 826 patients with IPF, when the patients are segregated according to % FVC (FIG. 1A), % DLco (FIG. 1B), time since IPF diagnosis (FIG. 1C) and FEV1/FVC ratio (FIG. 1D).

FIGS. 2A-B depict a re-analysis of data on mean change in FVC in pirfenidone-treated vs. placebo-treated groups from Study 1 and Study 2. FIG. 2A displays results for the patient population selected using the original Intention To Treat criteria. FIG. 2B displays results for the patient population selected using the novel criteria described in Example 2.

FIGS. 3A-B depict the data from FIG. 2B, separated into Study 1 (FIG. 3A) and Study 2 (FIG. 3B).

FIGS. 4A-B depict a re-analysis of data on mean change in 6 minute walk distance (6MWD) in pirfenidone-treated vs. placebo-treated groups from Study 1 and Study 2. FIG. 4A displays results for the patient population selected using the original Intention To Treat criteria. FIG. 4B displays results for the patient population selected using the novel criteria described in Example 2.

FIGS. 5A-B depict the data from FIG. 4B, separated into Study 1 (FIG. 5A) and Study 2 (FIG. 5B).

### DETAILED DESCRIPTION OF THE INVENTION

Prior patent applications relating to the use of pirfenidone in IPF patients include WO-2007/064738, WO-2007/038315, WO-2008/077068, WO-2010/056294, PCT/US2010/058935, and PCT/US2010/058943, each of which is incorporated by reference herein in its entirety.

The invention generally relates to improved uses and methods of administering pirfenidone to a patient in need of pirfenidone therapy, and to methods of preparing or packaging pirfenidone medicaments, containers, packages and kits. In any of the aspects or embodiments, the patient may have pulmonary fibrosis, such as idiopathic pulmonary fibrosis (IPF), and the medicament is for treatment of pulmonary fibrosis, or IPF.

A selected group of IPF patients that are more likely to experience FVC decline and disease progression over a period of a year has been identified. Their greater rate of progression, as reflected by a greater rate of decrease in respiratory parameters such as FVC, correlates with a greater relative magnitude of pirfenidone treatment effect.

Data described in the examples show that IPF patients with the following criteria experience a greater FVC decline, as measured by % FVC change from baseline or proportion of patients with 10% or greater % FVC decline at a specified timepoint, compared to patients that do not meet the criteria. The examples show that patients with the following criteria also exhibited a greater observed pirfenidone treatment effect on alleviating the extent of FVC decline compared to patients that do not meet the criteria.

(a) % FVC 50% -90%

(b) FEV1/FVC ratio >0.80

(c) Time since IPF diagnosis >0.5 years and <48 months

The invention provides a method of treating pulmonary fibrosis, optionally IPF, comprising (a) selecting a patient that exhibits (i) percent of predicted forced vital capacity volume (% FVC) of about 90% or less, or (ii) ratio of forced expiratory volume in one second (FEV1) to forced vital capacity volume (FVC) of about 0.80 or greater, or both, and (b) administering a therapeutically effective amount of pirfenidone.

In a related aspect, the invention provides pirfenidone for use in treating pulmonary fibrosis in a patient that exhibits (i) percent of predicted forced vital capacity volume (% FVC) of about 90% or less or (ii) ratio of forced expiratory volume in one second (FEV1) to forced vital capacity volume (FVC) of about 0.80 or greater, or both.

In a further related aspect, the invention provides use of pirfenidone in preparation of a medicament for treating pulmonary fibrosis in a patient that exhibits (i) percent of predicted forced vital capacity volume (% FVC) of about 90% or less or (ii) ratio of forced expiratory volume in one second (FEV1) to forced vital capacity volume (FVC) of about 0.80 or greater, or both.

Optionally, in some or any of these embodiments, % FVC ranges from about 50% to about 90%. In some or any embodiments, the patient has been diagnosed with pulmonary fibrosis, optionally IPF, for at least six months, and optionally less than 48 months. In some or any embodiments, optionally the patient is also selected to exhibit a percent of diffusing capacity (% DLco) of about 90% or less, for example, ranging from 30% to 90%, or 30% to 60%, inclusive of both endpoints. In some or any embodiments, the FEV1/FVC ratio is greater than 0.9. In some or any embodiments, the % FVC is less than 80%, 70%, or 60%. In some or any embodiments, the % Dlco is less than 90%, 80%, 70%, 60%, or 50%, or less than 40%. In most cases the patient is diagnosed with IPF through a High Resolution Computed Tomography (HRCT) scan, optionally with confirmation through surgical lung biopsy.

### Agents Administered in Disclosed Methods to Disclosed Select Patient Population

The agent administered to the selected patient population according to the uses described herein can be one or more of steroids (including but not limited to prednisolone), cytotoxic agents (including but not limited to azathioprine and cyclophosphamide), bardoxolone, LPA antagonists, for example LPA1 (including but not limited to AM152); Torisel (temsirolimus); PI3K inhibitors; pentraxin (including but not limited to Pentraxin-2 (PTX-2 or PRM-151)); MEK inhibitors (including but not limited to ARRY-162 and ARRY-300); p38 inhibitors; PAI-1 inhibitors (including but not limited to Tiplaxtinin); agents that reduce the activity of transforming growth factor-beta (TGF-β) (including but not limited to pan TGF-β neutralizing antibodies, such as GC-1008 (Genzyme/Medlmmune); anti-TGF-β2 mAbs, such as lerdelimumab (CAT-152; Trabio, Cambridge Antibody); anti-TGF-β1 antibodies, such as metelimumab(CAT-192,Cambridge Antibody); small molecule TGF-βR1 inhibitors, such as LY-2157299 (Eli Lilly); ACU-HTR-028 (Opko Health)) including antibodies that target one or more TGF-β isoforms, inhibitors of TGF-β receptor kinases TGFBR1 (ALKS) and TGFBR2, and modulators of post-receptor signaling pathways; modulators of chemokine receptor signaling; endothelin receptor antagonists including inhibitors that target both endothelin receptor A and B and those that selectively target endothelin receptor A (including but not limited to ambrisentan; avosentan; bosentan; clazosentan; darusentan; BQ-153; FR-139317, L-744453; macitentan; PD-145065; PD-156252; PD163610;PS-433540; S-0139; sitaxentan sodium; TBC-3711; zibotentan); agents that reduce the activity of connective tissue growth factor (CTGF) (including but not limited to FG-3019, FibroGen), and also including other CTGF-neutralizing antibodies, such as FG-3019; matrix metalloproteinase (MMP) inhibitors (including but not limited to MMPI-12, PUP-1 and tigapotide triflutate, and doxycycline, marimastat, and cipemastat); agents that reduce the activity of epidermal growth factor receptor (EGFR) including but not limed to erlotinib, gefitinib, BMS-690514, cetuximab , antibodies targeting EGF receptor, inhibitors of EGF receptor kinase, and modulators of post-receptor signaling pathways; agents that reduce the activity of platelet derived growth factor (PDGF) (including but not limited to Imatinib mesylate (Novartis)) and also including PDGF neutralizing antibodies, antibodies targeting PDGF receptor (PDGFR), inhibitors of PDGFR kinase activity, and post-receptor signaling pathways; agents that reduce the activity of vascular endothelial growth factor (VEGF) (including but not limited to axitinib, bevacizumab, BIBF-1120, CDP-791, CT-322, IMC-18F1, PTC-299, and ramucirumab) and also including VEGF-neutralizing antibodies, antibodies targeting the VEGF receptor 1 (VEGFR1, Flt-1) and VEGF receptor 2 (VEGFR2, KDR), the soluble form of VEGFR1 (sFlt) and derivatives thereof which neutralize VEGF, and inhibitors of VEGF receptor kinase activity; inhibitors of multiple receptor kinases such as BIBF-1120 which inhibits receptor kinases for vascular endothelial growth factor, fibroblast growth factor, and platelet derived growth factor; agents that interfere with integrin function (including but not limited to STX-100 and IMGN-388) and also including integrin targeted antibodies; agents that interfere with the pro-fibrotic activities of IL-4 (including but not limited to AER-001, AMG-317, APG-201, and sIL-4Rα) and IL-13 (including but not limited to AER-001, AMG-317, anrukinzumab, CAT-354, cintredekin besudotox, MK-6105, QAX-576, SB-313, SL-102, and TNX-650) and also including neutralizing antibodies to either cytokine, antibodies that target IL-4 receptor or IL-13 receptor, the soluble form of IL-4 receptor or derivatives thereof that is reported to bind and neutralize both IL-4 and IL-13, chimeric proteins including all or part of IL-13 and a toxin particularly pseudomonas endotoxin, signaling though the JAK-STAT kinase pathway; agents that interfere with epithelial mesenchymal transition including inhibitors of mTor (including but not limited to AP-23573 or rapamycin); agents that reduce levels of copper such as tetrathiomolybdate; agents that reduce oxidative stress including N-acetyl cysteine and tetrathiomolybdate; and interferon gamma. Also contemplated are agents that are inhibitors of phosphodiesterase 4 (PDE4) (including but not limited to Roflumilast); inhibitors of phosphodiesterase 5 (PDES) (including but not limited to mirodenafil, PF-4480682, sildenafil citrate, SLx-2101, tadalafil, udenafil, UK-369003, vardenafil, and zaprinast); or modifiers of the arachidonic acid pathway including cyclooxygenase and 5-lipoxegenase inhibitors (including but not limited to Zileuton). Further contemplated are compounds that reduce tissue remodeling or fibrosis including prolyl hydrolase inhibitors (including but not limited to 1016548, CG-0089, FG-2216, FG-4497, FG-5615, FG-6513, fibrostatin A (Takeda), lufironil,P-1894B, and safironil) and peroxisome proliferator-activated receptor (PPAR)-gamma agonists (including but not limited to pioglitazone and rosiglitazone). Agents also include an agent selected from BG-12, chemokine activity modulators (including but not limited to CNTO 888, an antibody targeting CCL2), Lys1 oxidase inhibitors (including but not limited to AB0024/GS-6624, an antibody targeting human lysyl oxidase-like 2), NOX4 inhibitors (including but not limited to GKT137831, a selective NOX 1/4 inhibitor), angiotensis II receptor antagonists (including but not limited to lorsartan), an LPA1/LPA3 antagonist (including but not limited to SAR-100842), and a deuterated pirfenidone (as described e.g., in W009/035598 and have one to fourteen deuterium atoms replacing a hydrogen atom in pirfenidone).

For example for LPA1 receptor antagonists, the agent can be

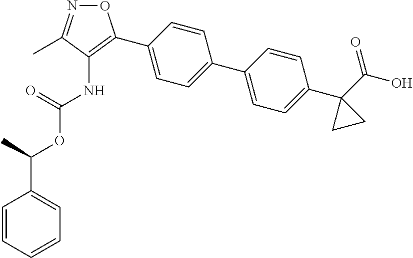 [SMILES: Cc1noc(-c2ccc(-c3ccc(C4(C(=O)O)CC4)cc3)cc2)c1NC(=O)O[C@H](C)c1ccccc1]

or one or more of (4′-{3-Methyl-4-[1-(2-trifluoromethyl-phenyl)-ethoxycarbonylamino]-isoazol-5-yl}-biphenyl-4-yl)-acetic acid (Compound 1); (4′-{3-Methyi-4-[1-(3-trifluoromethyl-phenyl)-ethoxycarbonoylamino]-isoazol-5-yl}-biphenyl-4-yl)-acetic acid (Compound 2); (4′-{4-[1-(2,4-Dichloro-phenyl)-ethoxycarbonylamino]-3-methyl-isoxazol-5-yl}-biphenyl-4-yl)-acetic acid (Compound 3); (4′-{4-[1-(2-Fluoro-phenyl)-ethoxycarbonbylamino]-3-methyl-isoxazol-5-yl}-biphenyl-4-yl)-aceti acid (Compound 4); (4′-{4-[1-(3-Bromo-phenyl)-ethoxycarbonylamino]-3-methyl-isoxazol-5-yl}-biphenyl-4-yl)-acetic acid (Compound 5); (4′-{4-[1-(2-Methoxy-phenyl)-ethoxycarbonylamino]-3-methyl-isoxazol-5-yl}-biphenyl-4-yl)-acetic acid (Compound 6); (4′-{4-[1-(2-Chloro-phenyl)-ethoxycarbonylamino]-3-methyl-isoxazol-5-yl}-6-methoxy-biphenyl-3-yl)-acetic acid (Compound 7); 4′-{4-[1-(2-Chloro-phenyl)-ethoxycarbonylamino]-3-methyl-isoxazol-5-yl}-biphenyl-4-carboxylic acid (Compound 8); 4′-{4-[1-(2-Chloro-pphenyl)-ethoxycarbonylamino]-3-methyl-isoxazol-5-yl}-bipehnyl-2-carboxylic acid (Compound 9); (4-{4-[1-(2-Chloro-phenyl)-ethoxycarbonylamino]-3-methyl-isoxazol-5-yl}-biphenyl-2-yl)-acetic acid (Compound 10); (4′-{4-[1-(2-Chloro-phenyl)-ethoxycarbonylamino]-3-methyl-isoxazol-5-yl}-biphenyl-4-yl)-acetic acid (Compound 11); (4′-{4-[1-(2-Chloro-phenyl)-ethoxycarbonylamino]-3-methyl-isoxazol-5-yl}-biphenyl-3-yl)-acetic acid (Compound 2); 3-(4′-{4-[1-(2-Chloro-phenyl)-ethoxycarbonylamino]-3-methyl-isoxazol-5-yl}-biphenyl-4-yl)-propionic acid (Compound 13); (4′-{4-[1-(2-Chloro-phenyl)-ethoxycarbonylamino]-3-methyl-isoxazol-5-yl}-6-fluoro-biphenyl-3-yl)- acetic acid (Compound 14); (4′-{4-[1-(2-Chloro-phenyl)-ethoxycarbonylamino]-3-methyl-isoxazol-5-yl}-4-fluoro-biphenyl-3-yl)-acetic acid (Compound 15); (4′-{4-[1-(2-Chloro-phenyl)-ethoxycarbonylamino]-3-methyl-isoxazol-5-yl)-biphenyl-4yl)-acetic acid methyl ester (Compound 16); 2-(4′-{4-[1-(2-Chloro-phenyl)-ethoxycarbonylamino]-3-methyl-isoxazol-5.-yl}-biphenyl-4-yl)-propionic acid ethyl ester (Compound 17); 2-(4′-(4-[1-(2-Chloro-phenyl)-ethoxycarbonylamino]-3-methyl-isoxazol-5-yl}-biphenyl-4-yl)--propionic acid. (Compound 18); 2-(4′-{4-[1-(2-Chloro-phenyl)-ethoxycarbonylamino]-3-methyl-isoxazol-5-yl }-biphenyl-4-yl)-2-methyl-propionic acid (Compound 19); 2-(4′-{4-[1-(2-Fluora-phenyl)-ethoxycarbonylamino]-3-methyl-isoxazol-5-yl}-biphenyl-4-yl)-propionic acid (Compound 20); 4-(4′-{4-[1-(2-Chloro-phenyl)-ethoxycarbonylamino]-3-methyl-isoxazol-5-yl)-biphenyl-5-yl}-biphenyl-4-yl)-butyric acid (Compound 21); 4′-{4-[1-(2-Chloro-phenyl)-ethoxycarbonylamino]-3-methyl-isoxazol-5-yl)-biphenyl-3-carboxylic acid. (Compound 22); (4′-{4-[1-(4-Chloro-2-fluoro-phenyl)-ethoxycarbonylamino]-3-methyl-isoazol-5-yl}-biphenyl-4-yl)-acetic acid (Compound 23); (4′-{4-[(R)-1-(2-Fluoro-phenyl)-ethoxycarbonylamino]-3-methyl-isoxazol-5}-biphenyl-4-yl)-acetic acid (Compound 24); (4′-{4-[(R)-1-(2-Fluoro-phenyl)-ethoxycarbonylamino]-3-methyl-isoxazol-5-yl}-2′-methyl-biphenyl-4-yl)-acetic acid (Compound 25); 2-(4′-[1-(2-Fluro-phenyl)-ethoxycarbonylamino]-3-methyl-isoxazol-5-yl}-biphenyl-4-yl)-2-methyl-propionic acid (Compound 26); (4′-{4-[(R)-1-(2-Chloro-phenyl)-ethoxycarbonylamino]-3-methyl-isoxazol-5-yl}-biphenyl-4-yl)-acetic acid (Compound 27); 2-(4′-{4-[(R)-1-(2-Chloro-pehnyl)-ethoxycarbonylamino]-3-methyl-isoxazol-5-yl}-biphenyl-4-yl)-2-methyl-propionic acid (Compound 28); 2-(4′-{4-[(R)-1-(2-Fluoro-phenyl)-ethoxycarbonylamino]-3-methyl-isoxazol-5-yl}-biphenyl-4-yl)-2-methyl-propionic acid (Comp id 29); 2-(4′-{4-[(R)-1-(2-Fluoro-phenyl)-ethoxycarbonylamino]-3-methyl-isoxazol-5-yl}-biphenyl-4-yl)-propionic acid (Compound 30); 2-(4′-{4-[(R)-1-(2-Chloro-phenyl)-ethoxycarbonylamino]-3-methyl-isoxazol-5-yl}-biphenyl-4-yl)-propionic acid (Compound 31); (4′-{4-[1-(2,6-Dichloro-phenyl)-ethoxycarbonylamino]-3-methyl-isoxazol-5-yl) -biphenyl-4-yl)-acetic acid (Compound 32); 2-(4′-{4-[(R)-1-(2-Fluoro-phenyl)-ethoxycarbonylamino]-3-methyl-isoxazol-5-yl}-2′-methyl-biphenyl-4-yl)-propionic acid (Compound 33); (4′-{4-[(S)-1-(2-Fluoro-phenyl)-ethoxycarbonylamino]-3-methyl-isoxazol-5-yl}-biphenyl-4-yl)-sectice acid (Compound 34) (4′-{4-[(S)-1-(2-Chloro-phenyl)-ethoxycarbonylamino]-3-methyl-isoxazol-5-yl}-biphenyl-4-yl)-acetic acid (Compound 35); {4′-[4-(2-Chloro-benzyloxycarbonylamino)-3-methyl -isoxazol-5-yl]-biphenyl-4-yl}-acetic acid (Compound 36); {4′-[3-Methyl-4-((R)-1-phenyl-ethoxycarbonylamino)-isoxazol-5-yl]-biphenyl-4-yl}-acetic acid (Compound 37); (4′-{4-[1-(2,3-Difluoro-phenyl)-ethoxycarbonylamino]-3-methyl-isoxazol-5-yl}-biphenyl-4-yl)-acetic acid (Compound 38); (4′-{4-[1-(2,4-Difluor)-phenyl)-ethoxycarbonylamino]-3-methyl-isoxazol-5-yl}-biphenyl-4-yl)-acetic acid (Compound 39); (4′-[4-[1-(2-Fluoro-4-methoxy-phenyl)-ethoxycarbonylamino]-3-methyl-isoxazol-5-yl}-biphenyl-4-yl)-acetic acid (Compound 40); (4′-{4-[1-(2,5-Difluoro-phenyl)-ethoxycarbonylamino]-3-methyl-isoxazol-5-yl}-biphenyl-4-yl)-acetic acid (Compound 41); (4′-{4-[1-(2,6-Difluoro-phenyl)-ethoxycarbonylamino]-3-methyl-isoxazol-5-yl}-biphenyl-4-yl)-acetic acid (Compound 42); {4′-[3-Methyl-4-((R)-1-phenyl-ethoxycathonylamino)-isoxazol-5-yl]-biphenyl-3-yl}-acetic acid (Compound 43); 4′-[3-Methyl-4-((R)-1-phenyl-ethoxycarbonylamino)-isoxazol-5-yl]-biphenyl-4-carboxylic acid (Compound 44); {4′-[3-Methyl-4-((R)-1-phenyl-ethoxycarbonylamino)-isoxazol-5-yl]-biphenyl-2-yl}-acetic acid (Compound 45) {4′-[3-Methyl-4-((R)-1-o-tolyl-ethoxycarbonylamino)-isoxazol-5-yl]-biphenyl-4-yl}-acetic acid (Compound 46); 2-(4′-{4-[((R,R)-1-(2-Chloro-phenyl)-ethoxycarbonylamino]-3-methyl-isoxazol-5-yl}-biphenyl-4-yl)-propionic acid (Compound 47); 2-(4′-{4-(R,S)-1-(2-Chloro-phenyl)-ethoxycarbonylamino]-3-methyl-isoxazol-5-yl}-biphenyl-4-yl)-propionic acid (Compound 48); (3′-Chloro-4′-{4-[(R)-1-(2-chloro-phenyl)-ethoxycarbonylamino]-3-methyl-isoxazol-5-yl}-biphenyl-4-yl)-acetic acid (Compound 49); 2-(4′-{4-[(R)-1-(2-Chloro-phenyl)-ethoxycarbonylamino]-3-methyl-isoxazol-5-yl}-biphenyl-4-yl)-butyric acid (Compound 50); (2′-Chloro-4′-[(R)-1-(2-chloro-phenyl)-ethoxycarbonylamino]-3-methyl-isoxazol-5-yl)-biphenyl-4-yl)-acetic acid (Compound 51); (4′-{4-[(R)-1-(2-Chloro-phenyl)-ethoxycarbonylamino]-3-methyl-isoxazol-5-yl}-2′-fluoro-biphenyl-4-yl)-acetic acid (Compound 52); 4′-{4-[(R)-1-(2-Chloro-phenyl)-ethoxycarbonylamino]-3-methyl-isoxazol-5-yl}-biphenyl-4-carboxylic acid (Compound 53; (4′-{4-[(R)-1-(3,5-Dibromo-phenyl)-ethoxycarbonylamino]-3-methyl-isoxazol-5-yl}-biphenyl-4-yl)-acede: acid (Compound 56); {4′-[3-Methyl-4-((S)-1-phenyl-ethoxycarbonylamino)-isoxazol-5-yl]-biphenyl-4-yl}-acetic acid (Compound 57); (4′-{4-[(R)-1-(3-Hydroxy-phenyl)-ethoxycarbonylamino]-3-methyl-isoxazol-5-yl}-biphenyl-4-yl)-acetic acid (Compound 58); {4′-[3-Methyl-4-(1-phenyl-ethoxycarbonylamino)-isoxazol-5-yl]-biphenyl-4yl}-acetic add (Compound 59); [5-(4′-Cyanomethyl-biphenyl-4-yl)-3-methyl-isoxazol-4-yl]-carbamic acid (R)-1-(2-chloro-phenyl)-ethyl ester (Compound 61); [5-(4′-Cyanomethyl-biphenyl-4-yl)-3-methyl-isoxazol-4-yl]-carbamic acid (R)-1-(2-fluoro-phenyl)-ethyl ester (Compound 62) {3-Methyl-5-[4′-(2H-tetrazol-5-ylmethyl)-biphenyl-4-yl]-isoxazol-4-yl}-carbamic acid (R)-1-(2-fluoro-phenyl)-ethyl ester (Compound 63) {3-Methyl-5-[4′-(2H-tetrazol-5-ylmethyl)-biphenyl-4-yl]-isoxazol-4-yl}-carbamic acid (R)-1-(2-chloro-phenyl)-ethyl ester (Compound 64); [5-(4′-Carbaminidoylmethyl-biphenyl-4-yl)-3-methyl-isoxazol-4-yl]-carbamic acid (R)-1-(2-fluoro-phenyl)-ethyl ester (Compound 65); {5-[4′-(2-Acetylamino-2-imino-ethyl)-biphenyl-4-yl]-3-methyl-isoxazol-4-yl}-carbamic acid (R)-1-(2-fluoro-phenyl)-ethyl ester (Compound 66); and 2-(2-{4′-[3-Methyl-4-((R)-1-phenyl-ethoxycarbonylamino)-isoxazol-5-yl]-biphenyl-4-yl}-acetylamino)-ethansulfonic acid

In particular, the LPA1 receptor antagonist can have a structure of any one of formulae (I), (Ia), (II), (IIIa), (III), (IIIa), (IV), and (V) as disclosed in WO 2011/041462; a structure of any one of formulae (I), (II), and (III) as disclosed in WO 2010/68775; a structure of formula (I) as disclosed in US 2010/311799; a structure of formula (I) as disclosed in WO 2010/141761; a structure of any one of formulae (I), (II), (III), (IV) and (IV) as disclosed in WO 2010/141768; a structure of formula (I) as disclosed in US 2010/152257; a structure of any one of formulae (I), (II) and (III) as disclosed in WO 10/77882; a structure of formula (I) as disclosed in WO 10/77883; a structure of formula (I) as disclosed in US 2011/0082164; a structure of any one of formulae (I) and (II), as disclosed in WO 11/041461; a structure of any one of compounds 1-79 or formula (I) as disclosed in US 2011/0082181; a structure of any one of formulae (I), (II), (III), (IV), (V), (VI), and (VI) as disclosed in WO 2011/041694; a structure of formula (I) as disclosed in WO 11/041729; structure of any one of formulae (I), (II), (III), (IV), and (V) as disclosed in WO 11/017350, each of which is incorporated by reference in its entirety. These and related LPA1 receptor antagonists, and methods of synthesizing them, are disclosed generally in the following patent publications: WO 2010/68775; US 2010/311799; WO 2010/141761; WO 2010/141768; US 2010/152257; WO 2010/77883; WO 2010/77882, US 2011/82164; WO 2011/41461; WO 2011/41462; US 2011/82181; WO 2011/41694; WO 2011/41729; WO 2011/17350, each of which is incorporated by reference in its entirety.

Additional LPA1 receptor antagonists contemplated include compounds of formulae (1), (2) and (5), and in particular compounds 101-169, as disclosed in U.S. Pat. No. 6,964,975 and US Patent Publication No. 2003/114505, each of which is incorporated by reference in its entirety. A specific compound from this family is

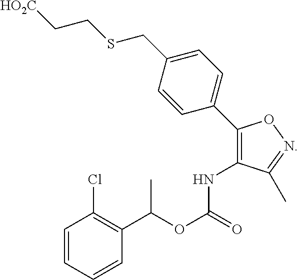 [SMILES: Cc1noc(-c2ccc(CSCCC(=O)O)cc2)c1NC(=O)OC(C)c1ccccc1Cl]

Still other LPA receptor antagonists contemplated include compounds disclosed in U.S. Pat. No. 7,517,996, and in particular a compound having a structure of formula (I), which is incorporated by reference in its entirety.

Still other LPA receptor antagonists contemplated include compounds disclosed in U.S. Pat. No. 7,288,558, and in particular compounds having a structure of formula (I), which is incorporated by reference in its entirety.

Also contemplated are agents that are PG D₂ modulators, such as compounds having a structure of any one of formulae (I), (II), (III), (IV), (V), (VI), (VII), (VIII), and (IX), as disclosed in US 2011/0098302 or structure of formula (I), as disclosed in US 2011/0098352, each of which is incorporated by reference in its entirety.

Also contemplated are the following agents or classes of agents: one or more of nitric oxide (e.g., inhaled nitric oxide), a vitamin E and pentoxifylline combination (e.g., PTL-202 from Pacific Therapeutics), PXS25, desatinib (a multiple kinase inhibitor), PI3K/mTor dual inhibitor (e.g., BAY806946, XL765, GDC0980, GSK2126458, BEZ235, BGT226, PF04691502, PK1587, and/or SF1126), PI3K inhibitor (e.g., XL147, GDC0941, BKM120, PX866, ZSTK474, BYL719 (PI3K alpha), AMG319 (PI3K delta), CAL101 (PI3K delta), and/or GDC0032), 5-HT2A/B receptor antagonists (e.g., terguride), telomerase activator (e.g., TAT153), modulators (e.g., reducers) of chemokine activity (e.g., CNTO 888, an antibody that targets CCL2), Lys1 oxidase inhibitors (e.g., AB0024/GS-6624, a humanized mAb targeting human lysyl oxidase-like 2), NOX4 inhibitor (e.g., GKT137831, a selective Nox 1/4 inhibitor), angiotensin II receptor antagonist (e.g., lorsartan), an anti αᵥβ₆ integrin agent, and pentraxin (e.g., serum amyloid P, PTX-2, or PRM-151).

Also contemplated are agents that are pirfenidone analogs, such as compounds having a structure of any one of formulae (I), (II), (III), (IV), and (V), as disclosed in WO 10/085805, the disclosure of which is incorporated by reference in its entirety. The synthesis of the pirfenidone analog compounds disclosed in WO 10/085805 are further described in U.S. Patent Publication No. 2007/0049624 (US national stage of WO 05/0047256), International Publication No. WO 03/068230, WO 08/003141, WO 08/157786, or in U.S. Pat. Nos. 5,962,478; 6,300,349; 6,090,822; 6,114,353; Re. 40,155; 6,956,044; or 5,310,562, each of which is incorporated by reference in its entirety.

The pirfenidone analogs disclosed in WO 10/085805, have structures of formulae (I), (II), (III), (IV), or (V):

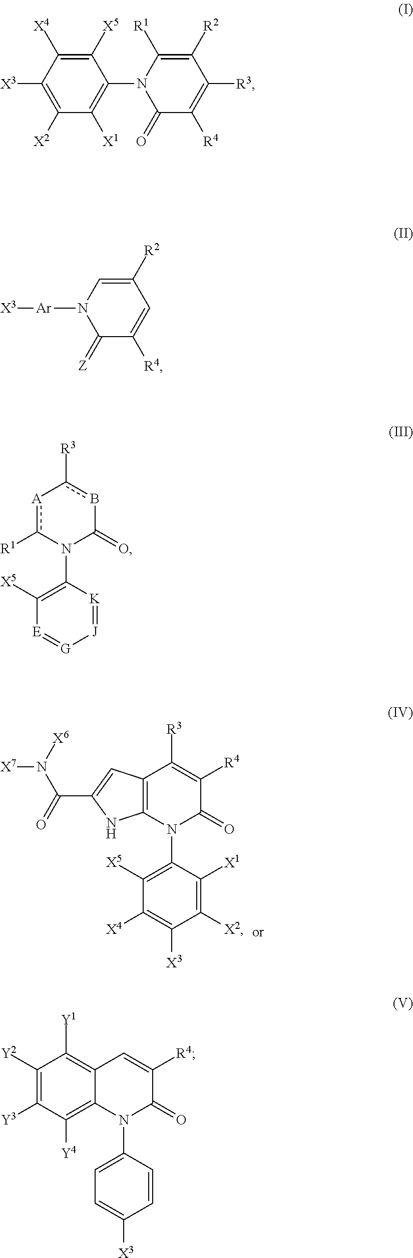

wherein A is N or CR²; B is N or CR⁴; E is Nor CX⁴; G is N or CX³; J is N or CX²; K is N or CX¹; a dashed line is a single or double bond, R¹, R², R³, R⁴, X¹, X², X³, X⁴, X⁵, Y¹, Y², Y³, and Y⁴ are independently selected from the group consisting of H, deuterium, C₁-C₁₀ alkyl, C₁-C₁₀ deuterated alkyl, substituted C₁-C₁₀ alkyl, C₁-C₁₀ alkenyl, substituted C₁-C₁₀ alkenyl, C₁-C₁₀ thioalkyl, C₁-C₁₀ alkoxy, substituted C₁-C₁₀ alkoxy, cycloalkyl, substituted cycloalkyl, heterocycloalkyl, substituted heterocycloalkyl, heteroalkyl, substituted heteroalkyl, aryl, substituted aryl, heteroaryl, substituted heteroaryl, halogen, hydroxyl, C₁-C₁₀ alkoxyalkyl, substituted C₁-C₁₀ alkoxyalkyl, C₁-C₁₀ carboxy, substituted C₁-C₁₀ carboxy, C₁-C₁₀ alkoxycarbonyl, substituted C₁-C₁₀ alkoxycarbonyl, CO-uronide, CO-monosaccharide, CO-oligosaccharide, and CO-polysaccharide; X⁶ and X⁷ are independently selected from the group consisting of hydrogen, aryl, substituted aryl, heteroaryl, substituted heteroaryl, cycloalkyl, substituted cycloalkyl, heterocycloalkyl, substituted heterocycloalkyl, alkylenylaryl, alkylenylheteroaryl, alkylenylheterocycloalkyl, alkylenylcycloalkyl, or X⁶ and X⁷ together form an optionally substituted 5 or 6 membered heterocyclic ring; Ar is pyridinyl or phenyl; and Z is 0 or S. In some embodiments, A is N or CR²; B is N or CR⁴; E is N, N⁺X⁴ or CX⁴; G is N, N⁺X³ or CX³; J is N, N⁺X² or CX²; K is N, N⁺X¹ or CX¹; a dashed line is a single or double bond, R¹, R², R³, R⁴, X¹, X², X³, X⁴, X⁵, Y¹, Y², Y³, and Y⁴ are independently selected from the group consisting of H, deuterium, optionally substituted C₁-C₁₀ alkyl, optionally substituted C₁-C₁₀ deuterated alkyl, optionally substituted C₁-C₁₀ alkenyl, optionally substituted C₁-C₁₀ thioalkyl, optionally substituted C₁-C₁₀ alkoxy, optionally substituted cycloalkyl, optionally substituted heterocycloalkyl, optionally substituted heteroalkyl, optionally substituted aryl, optionally substituted heteroaryl, optionally substituted amido, optionally substituted sulfonyl, optionally substituted amino, optionally substituted sulfonamido, optionally substituted sulfoxyl, cyano, nitro, halogen, hydroxyl, SO₂H₂, optionally substituted C₁-C₁₀ alkoxyalkyl, optionally substituted C₁-C₁₀ carboxy, optionally substituted C₁-C₁₀ alkoxycarbonyl, CO-uronide, CO-monosaccharide, CO-oligosaccharide, and CO-polysaccharide; X⁶ and X⁷ are independently selected from the group consisting of hydrogen, optionally substituted aryl, optionally substituted heteroaryl, optionally substituted cycloalkyl, optionally substituted heterocycloalkyl, optionally substituted alkylenylaryl, optionally substituted alkylenylheteroaryl, optionally substituted alkylenylheterocycloalkyl, optionally substituted alkylenylcycloalkyl, or X⁶ and X⁷ together form an optionally substituted 5 or 6 membered heterocyclic ring; and Ar is optionally substituted pyridinyl or optionally substituted phenyl; and Z is O or S.

The pirfenidone administered in the methods disclosed herein can be deuterated. The pirfenidone can be a mixture of deuterated forms of pirfenidone, a single deuterated form, or a mixture of deuterated form (or forms) and non-deuterated pirfenidone. Contemplated deuterated pirfenidone includes pirfenidone with 1, 2, 3, 4, 5, 6, 7, 8, 9, 10, 11, 12, 13, or 14 deuterium atoms. The phenyl ring of pirfenidone can be deuterated with 1, 2, 3, 4,or 5 deuterium atoms. Additionally or alternatively, the methyl of pirfenidone can be deuterated with 1, 2, or 3 deuterium atoms. Additionally or alternatively, the pyridone ring hydrogens can be substituted with 1, 2, 3, or 4 deuterium atoms. Multiple different deuterated pirfenidone forms and methods of synthesizing the various deuterated pirfenidone forms are disclosed in WO 09/035598, the disclosure of which is incorporated by reference in its entirety.

Combinations of one or more of the foregoing agents are also contemplated.

The terms “therapeutically effective amount,” as used herein, refer to an amount of a compound sufficient to treat, ameliorate, or prevent the identified disease or condition, or to exhibit a detectable therapeutic, prophylactic, or inhibitory effect. The effect can be detected by, for example, an improvement in clinical condition, or reduction in symptoms. The precise effective amount for a subject will depend upon the subject's body weight, size, and health; the nature and extent of the condition; and the therapeutic or combination of therapeutics selected for administration. Where a drug has been approved by the U.S. Food and Drug Administration (FDA) or a counterpart foreign medicines agency, a “therapeutically effective amount” optionally refers to the dosage approved by the FDA or its counterpart foreign agency for treatment of the identified disease or condition.

In any of the aspects or embodiments, the therapeutically effective amount of pirfenidone being administered may be a total daily dosage of at least about 1800 mg per day, or about 2400 mg or about 2403 mg per day, optionally administered in divided doses three times per day, with food. In any of the aspects of embodiments, the total daily dosage may be about 1200 to about 4000 mg per day, or about 1600 to about 3600 mg per day. In any of the aspects of the invention, the daily dosage may be administered in divided doses three times a day, or two times a day, or alternatively is administered in a single dose once a day. In any of the aspects of the invention, the pirfenidone may be administered with food. For example, the daily dosage of 2400 mg or 2403 mg pirfenidone per day may be administered as follows: 801 mg taken three times a day, with food.

Pirfenidone can be dosed at a total amount of about 50 to about 2400 mg per day. The dosage can be divided into two or three doses over the day. Specific amounts of the total daily amount of the therapeutic contemplated for the disclosed methods include about 50 mg, about 100 mg, about 150 mg, about 200 mg, about 250 mg, about 267 mg, about 300 mg, about 350 mg, about 400 mg, about 450 mg, about 500 mg, about 534 mg, about 550 mg, about 600 mg, about 650 mg, about 700 mg, about 750 mg, about 800 mg, about 850 mg, about 900 mg, about 950 mg, about 1000 mg, about 1050 mg, about 1068 mg, about 1100 mg, about 1150 mg, about 1200 mg, about 1250 mg, about 1300 mg, about 1335 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1869 mg, about 1900 mg, about 1950 mg, about 2000 mg, about 2050 mg, about 2100 mg, about 2136 mg, about 2150 mg, about 2200 mg, about 2250 mg, about 2300 mg, about 2350 mg, and about 2400 mg.

Dosages of pirfenidone can alternately be administered as a dose measured in mg/kg. Contemplated mg/kg doses of the disclosed therapeutics include about 1 mg/kg to about 40 mg/kg. Specific ranges of doses in mg/kg include about 20 mg/kg to about 40 mg/kg, or about 30 mg/kg to about 40 mg/kg.

Any of the criteria described herein also may be applied to patients suffering from pulmonary fibrosis diseases generally, including but not limited to idiopathic or usual interstitial pneumonia, autoimmune lung diseases, chronic obstructive pulmonary disease (COPD), inflammatory pulmonary fibrosis, fibrosis secondary to asthma; adult respiratory distress syndrome; pulmonary sarcosis; fibrosis secondary to lung cancer, fibrosis secondary to graft-versus-host reaction; fibrosis secondary to viral diseases, including influenza virus, Severe Acute Respiratory Syndrome (SARS).

In another aspect, a package or kit is provided comprising pirfenidone, optionally in a container, and a package insert, package label, instructions or other labeling including any of the criteria for patient selection described herein. The package insert, package label, instructions or other labeling may further comprise directions for treating IPF by administering pirfenidone, e.g., at a dosage of at least about 1800 mg per day, or a dosage of about 2400 mg or about 2403 mg per day.

In related aspect, the invention provides a method of preparing or packaging a pirfenidone medicament comprising packaging pirfenidone, optionally in a container, together with a package insert or package label or instructions including any of the foregoing information or recommendations.

In some embodiments, a method of treating IPF is disclosed comprising providing, selling or delivering any of the kits of disclosed herein to a hospital, physician or patient.

The invention will be more fully understood by reference to the following examples which detail exemplary embodiments of the invention. They should not, however, be construed as limiting the scope of the invention. All citations throughout the disclosure are hereby expressly incorporated by reference.

### EXAMPLES

### Example 1

### Identification of Predictors of Significant FVC Decline

Two multinational, randomized, double-blind, placebo-controlled Phase 3 trials, referred to herein as Study 1 and Study 2, were designed and performed to evaluate the safety and efficacy of pirfenidone in IPF patients with mild to moderate impairment in lung function. Both trials enrolled patients in North America, Europe and Australia with roughly 75% of the total 779 patients enrolled in North America.

Study 1 enrolled a total of 344 patients. Patients were randomized 1:1 to receive a total daily dose of 2403 mg pirfenidone, or placebo. Study 2 enrolled a total of 435 patients, and patients were randomized 2:2:1 to receive a total daily dose of 2403 mg pirfenidone, or placebo, or a total daily dose of 1197 mg pirfenidone, respectively. The total daily dose of pirfenidone was administered in three divided doses, three times per day.

Inclusion criteria included: (a) age 40-80 years of age; (b) clinical symptoms of IPF (dyspnea on exertion) for >3 months duration; (c) diagnosis of IPF within 48 months of randomization; (d) HRCT showing confident radiographic diagnosis of usual interstitial pneumonia (UIP), if surgical lung biopsy showing definite or probable UIP, HRCT criteria of probable UIP was sufficient; (e) if <50 years of age, open or video assisted thoracoscopic surgical (VATS) lung biopsy showing definite or probable UIP within 48 months of randomization and no features of alternative diagnosis on transbronchial biopsy or bronchoalveolar lavage (BAL) (if performed); (f) if ≧50 years of age, at least one of the following and absence of features that supported alternative diagnosis within 48 months of randomization: 1) open or VATS lung biopsy that showed definite or probable UIP; 2) transbronchial biopsy showing no alternative diagnosis; 3) BAL showing no alternative diagnosis. If the surgical lung biopsy or HRCT scans were ambiguous, the slides or HRCT images were evaluated by an adjudicator for a second opinion.

Exclusion criteria included: FEV1/FVC<0.7 after bronchodilator administration, bronchodilator response, RV>120%, history of significant environmental exposure known to cause pulmonary fibrosis, and diagnosis of connective tissue disease or other known explanation of interstitial lung disease (ILD).

Enrollment of both trials was completed in less than 13 months following randomization of the first patient into the program in late April 2006. Ninety-seven percent (97%) of all patients in the two studies who were living and had not received a lung transplant, completed their Week 72 study visit.

The primary endpoint of both trials was change in percent predicted Forced Vital Capacity (FVC) after 72 weeks of treatment evaluated with a nonparametric rank ANCOVA analysis. The pre-specified secondary endpoints monitored during the studies included: (a) Time to worsening of IPF, defined as time to acute IPF exacerbation, IPF-related death, lung transplant or respiratory hospitalization, whichever comes first; (b) Progression-free survival defined as time to the first occurrence of either of the following (as compared to the patient's baseline): (1) 10% absolute decline in percent predicted FVC, or (2) 15% absolute decline in percent predicted Hb-corrected DL(CO), or (3) death (in the case of FVC or DL(CO), the decline must be confirmed at two consecutive visits at least 6 weeks apart); (c) Categorical assessment of absolute change from Baseline to Week 72 in percent predicted FVC; (d) Change from Baseline to Week 72 in dyspnea measured by the University of California at San Diego Shortness-of-Breath Questionnaire (UCSD SOBQ); (e) Change from Baseline to Week 72 in the percent predicted Hb-corrected DL(CO); (f) Change from Baseline to Week 72 in the worst oxygen saturation by pulse oximetry (SpO2) measurement observed during the Six-Minute Walk Test; (g) Change from Baseline to Week 72 in distance walked in the Six-Minute Walk Test (6MWT).

After the studies were completed, a retrospective multivariate model of data identified predictors of greater FVC decline at 72 weeks and variables that have a statistical interaction with pirfenidone treatment (i.e. the magnitude of the pirfenidone treatment effect is different at different levels of these variables). The variables identifying this selected patient population with greater FVC decline and p-values are set forth in Table 1 below.

TABLE 1

| Variable | P-Value | Effect |
|---|---|---|
| Predictors | | Group w/ More FVC Decline |
| DL_{co} (median) | <0.001 | Low DL_{co} |
| FEV₁/FVC Ratio (0.80) | 0.012 | High FEV₁/FVC Ratio |
| BMI (median) | 0.005 | High BMI |
| | | Group with |
| Interaction with Treatment | | Greater Pirfenidone Effect |
| % FVC (90%) | 0.096 | FVC <90% |
| Time since Diagnosis | 0.043 | Time >0.5 years |
| of IPF (0.5 yrs) | | |

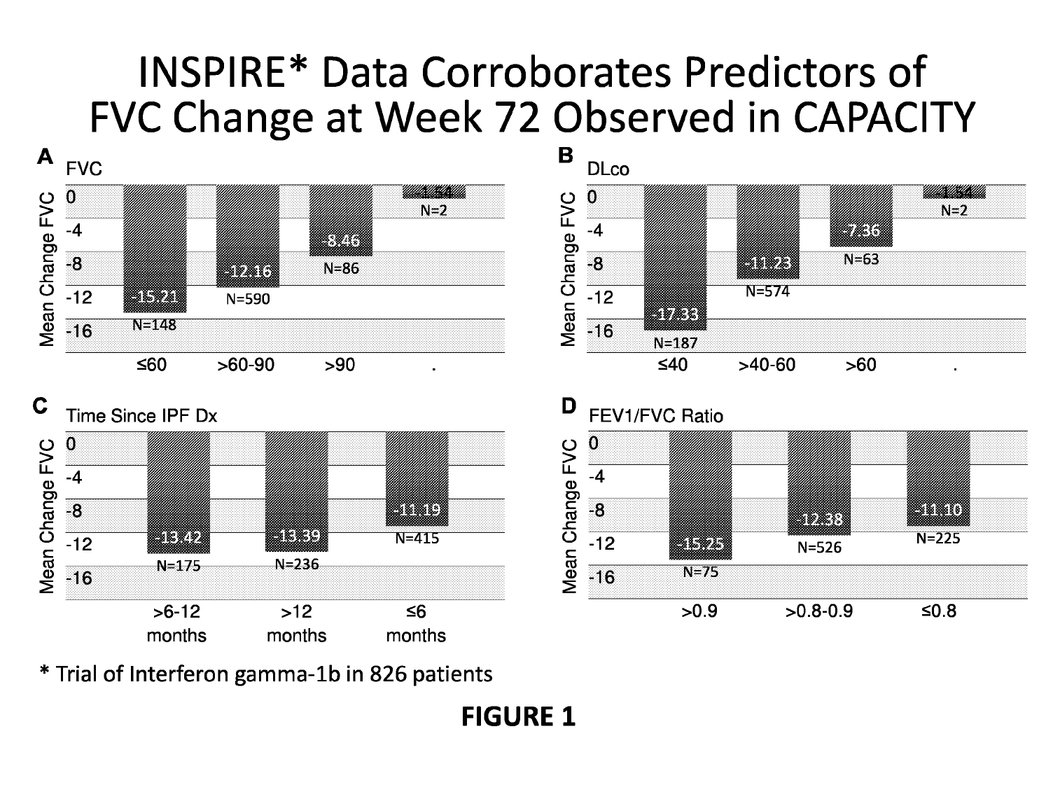

In a prior trial involving 826 patients, data regarding progression of FVC was collected as part of a randomized, double-blind, placebo-controlled Phase 3 study conducted from 2003-2007 that was designed to evaluate the safety and efficacy of Actimmune(R) in IPF patients with mild to moderate impairment in lung function. This data was also retrospectively analyzed for predictors of greater FVC decline. These analyses were based on both the placebo and active (i.e. Actimmune) treatment groups as this trial and others have demonstrated that Actimmune does not have an effect on these outcomes. Results of the analysis are set forth in FIGS. 1A-1D. Selecting patients according to each of % FVC (FIG. 1A), % DLco (FIG. 1B), time since IPF diagnosis (FIG. 1C) and FEV1/FVC ratio (FIG. 1D)produced respective patient populations that exhibited greater FVC decline from baseline.

The predictors of FVC decline were generally consistent across this 826-patient study, Study 1, Study 2, and the pooled population for Studies 1 and 2.

### Example 2

Pirfenidone Treatment Effect in Patient Population with Selected Criteria

Retrospective analysis of the data from Study 1 and Study 2 in Example 1 showed that the patient population with greater FVC decline also exhibited a greater observed pirfenidone treatment effect. The consistent augmentation of pirfenidone treatment effect across independent endpoints and studies strongly suggests these observations are at least directionally accurate.

Data from Study 1 and Study 2 were re-analyzed, selecting patients according to the following novel criteria instead of the original Intention To Treat (IIT) criteria:

(a) % FVC 50% -90%

(b) FEV1/FVC ratio >0.80

(c) Time since IPF diagnosis >0.5 years and <48 months

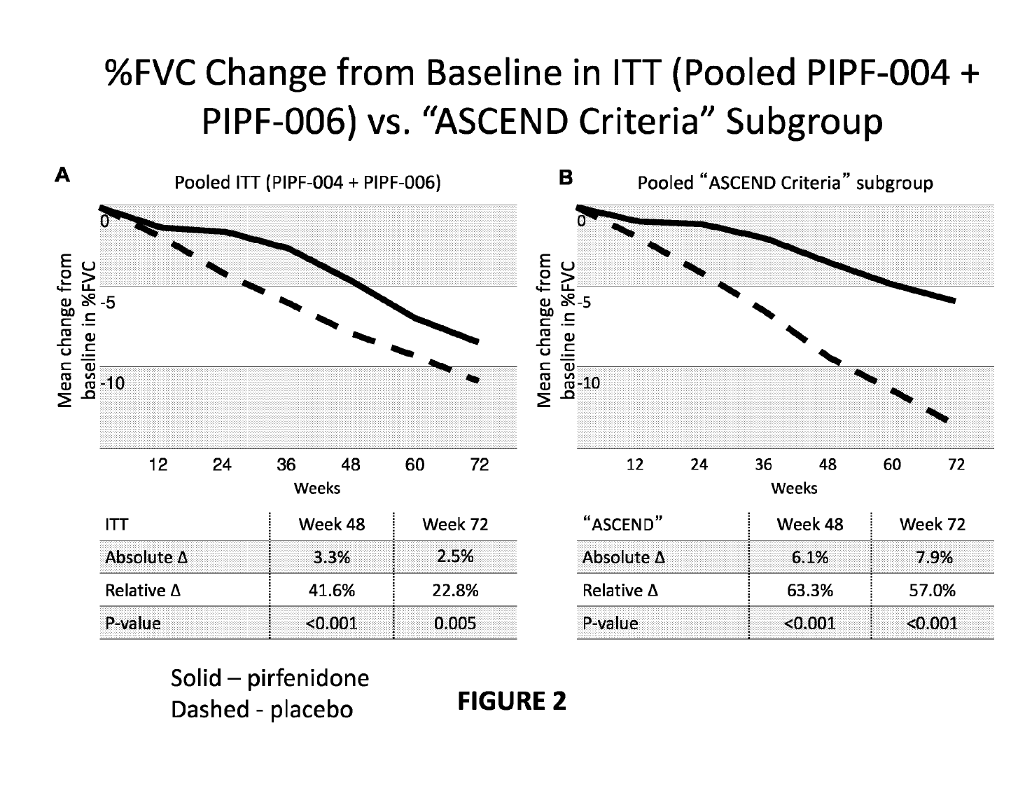

Mean change in FVC in pirfenidone-treated vs. placebo-treated groups from Study 1 and Study 2, for the original patient population selected using the ITT criteria, is shown in FIG. 2A. When the patient population is instead selected using the novel criteria above, the mean change in FVC in pirfenidone-treated vs. placebo-treated groups for the patient is shown in FIG. 2B. The same data for the original ITT criteria is shown in FIG. 2A. Patients in both pirfenidone-treated and placebo-treated groups experienced a decline in mean % FVC from baseline. When the original ITT criteria were used, the difference in mean change from baseline % FVC between pirfenidone-treated and placebo-treated groups was an absolute difference of 3.3% at week 48 (translating to a relative difference of 41.6%), p<0.001, and an absolute difference of 2.5% at week 72 (translating to a relative difference of 22.8%), p=0.005. When the novel criteria were used, the difference in mean change from baseline % FVC between pirfenidone-treated and placebo-treated groups was an absolute difference of 6.1% at week 48 (translating to a relative difference of 63.3%), p<0.001, and an absolute difference of 7.9% at week 72 (translating to a relative difference of 57.0%), p<0.001.

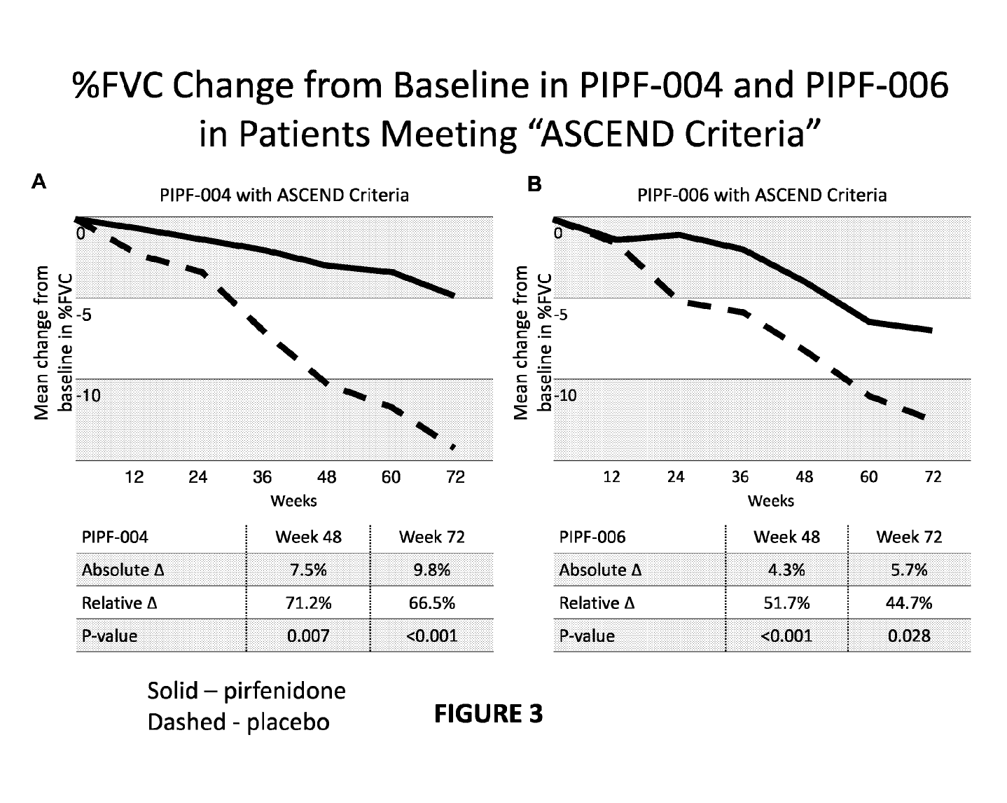

The data from FIG. 2B was further separated into Study 1 (FIG. 3A) and Study 2 (FIG. 3B).

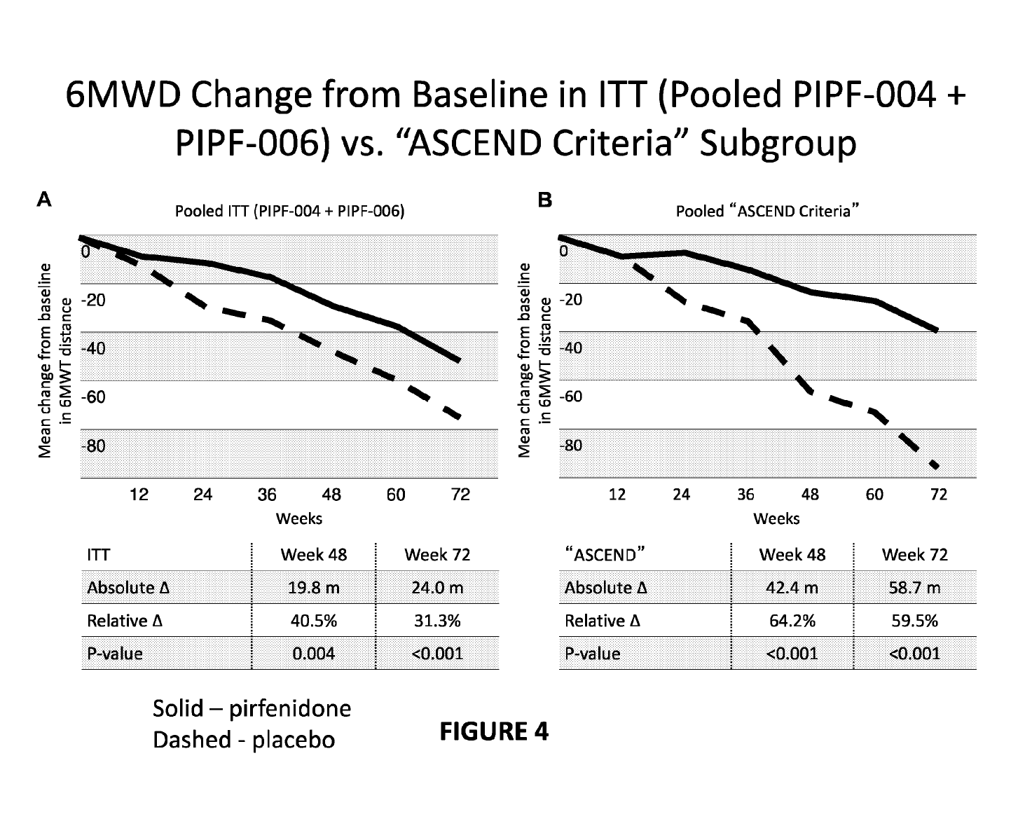

Mean change in 6 minute walk distance (6MWD) in pirfenidone-treated vs. placebo-treated groups from Study 1 and Study 2, for the original patient population selected using the ITT criteria, is shown in FIG. 4A. When the patient population is instead selected using the novel criteria above, the mean change in 6MWD in pirfenidone-treated vs. placebo-treated groups for the patient is shown in FIG. 4B. Patients in both pirfenidone-treated and placebo-treated groups experienced a decline in mean 6MWD from baseline.

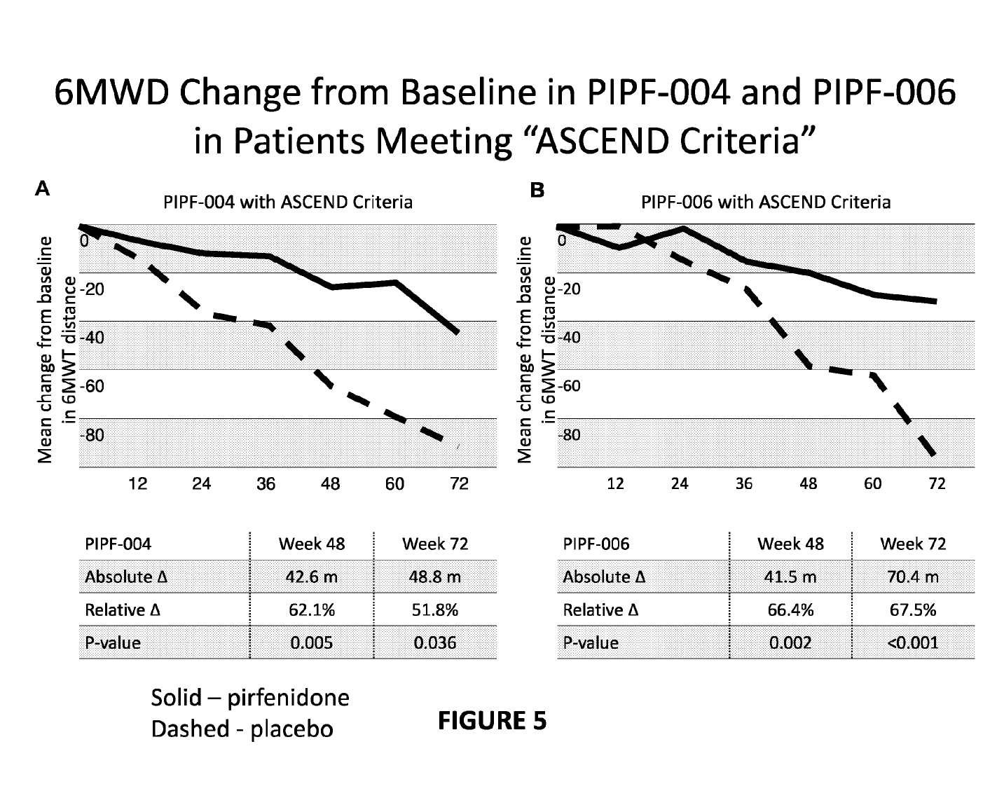

The data from FIG. 4B was further separated into Study 1 (FIG. 5A) and Study 2 (FIG. 5B).

Data on a variety of secondary endpoints at week 48 was re-analyzed using the novel criteria above to select the patient population. Relative difference in the endpoint for pirfenidone-treated vs. placebo-treated groups, from Study 1 and Study 2 pooled together, is shown below in Table 2 when the novel criteria or the original Intention To Treat (ITT) criteria are used to select the patient population.

TABLE 2

| Endpoint | Pooled ITT | | Novel criteria | |
|---|---|---|---|---|
| Change from Baseline | Relative Δ | P-value | Relative Δ | P-value |
| % FVC ≧10% decline | 39% | <0.001 | 60.4% | <0.001 |
| 6MWT | 41% | 0.004 | 64.2% | <0.001 |
| UCSD SOBQ (dyspnea) | −29% | 0.148 | −46.5% | 0.039 |
| % DLco | 16% | 0.195 | 36.9% | 0.015 |
| SpO₂ During 6MWT | 21% | 0.158 | 62.3% | <0.001 |
| Time to Event* | HR | P-value | HR | P-value |
| Progression-free survival | 0.53 | 0.002 | 0.31 | <0.001 |
| Worsening IPF | 0.80 | 0.383 | 0.75 | 0.469 |
| Survival | 0.42 | 0.025 | 0.16 | 0.007 |

While the present invention has been described in terms of various embodiments and examples, it is understood that variations and improvements will occur to those skilled in the art.

Examples of embodiments of the invention include:
1. A method of treating pulmonary fibrosis, optionally IPF, comprising (a) selecting a patient that exhibits (i) percent of predicted forced vital capacity volume (% FVC) of about 90% or less, or (ii) ratio of forced expiratory volume in one second (FEV1) to forced vital capacity volume (FVC) of about 0.80 or greater, or both, and (b) administering a therapeutically effective amount of pirfenidone.
2. Pirfenidone for use in treating pulmonary fibrosis in a patient that exhibits (i) percent of predicted forced vital capacity volume (% FVC) of about 90% or less or (ii) ratio of forced expiratory volume in one second (FEV1) to forced vital capacity volume (FVC) of about 0.80 or greater, or both.
3. Use of pirfenidone in preparation of a medicament for treating pulmonary fibrosis in a patient that exhibits (i) percent of predicted forced vital capacity volume (% FVC) of about 90% or less or (ii) ratio of forced expiratory volume in one second (FEV1) to forced vital capacity volume (FVC) of about 0.80 or greater, or both.
4. The method, pirfenidone or use of any of examples 1-3 and 10 wherein % FVC ranges from about 50% to about 90%.
5. The method, pirfenidone or use of any of examples 1-4 and 10 wherein the patient has been diagnosed with pulmonary fibrosis, optionally IPF, for at least six months, and optionally less than 48 months.
6. The method, pirfenidone or use of any of examples 1-5 and 10 wherein the patient exhibits a diffusion capacity (% DLco) ranging from about 30% to about 90%.
7. The method, pirfenidone or use of any one of examples 1-6 wherein the pirfenidone is administered at a total daily dosage of at least about 1800 mg.
8. The method, pirfenidone or use of any one of examples 1-7 wherein the pirfenidone is administered at a total daily dosage of about 2403 mg.
9. The method, pirfenidone or use of any one of examples 1-8 wherein the pirfenidone is administered to the patient three times per day, with food.
9A. The method, pirfenidone or use of any one of examples 1-9, wherein the pirfenidone comprises a deuterated pirfenidone as described herein.
10. A method of treating pulmonary fibrosis, optionally IPF, comprising (a) selecting a patient that exhibits (i) percent of predicted forced vital capacity volume (% FVC) of about 90% or less, or (ii) ratio of forced expiratory volume in one second (FEV1) to forced vital capacity volume (FVC) of about 0.80 or greater, or both, and (b) administering a therapeutically effective amount of an agent,
wherein the agent is selected from steroids (including but not limited to prednisolone), cytotoxic agents (including but not limited to azathioprine and cyclophosphamide), bardoxolone, LPA agonists (including but not limited to AM152); Torisel (temsirolimus); PI3K inhibitors; pentraxin or serum amyloid P (including but not limited to Pentraxin-2 (PTX-2 or PRM-151)); MEK inhibitors (including but not limited to ARRY-162 and ARRY-300); p38 inhibitors; PAI-1 inhibitors (including but not limited to Tiplaxtinin); agents that reduce the activity of transforming growth factor-beta (TGF-β) (including but not limited to GC-1008 (Genzyme/Medlmmune); lerdelimumab (CAT-152; Trabio, Cambridge Antibody); metelimumab(CAT-192,Cambridge Antibody,); LY-2157299 (Eli Lilly); ACU-HTR-028 (Opko Health)) including antibodies that target one or more TGF-β isoforms, inhibitors of TGF-β receptor kinases TGFBR1 (ALKS) and TGFBR2, and modulators of post-receptor signaling pathways; chemokine receptor signaling; endothelin receptor antagonists including inhibitors that target both endothelin receptor A and B and those that selectively target endothelin receptor A (including but not limited to ambrisentan; avosentan; bosentan; clazosentan; darusentan; BQ-153; FR-139317, L-744453; macitentan; PD-145065; PD-156252; PD163610;PS-433540; S-0139; sitaxentan sodium; TBC-3711; zibotentan); agents that reduce the activity of connective tissue growth factor (CTGF) (including but not limited to FG-3019, FibroGen), and including other CTGF-neutralizing antibodies; matrix metalloproteinase (MMP) inhibitors (including but not limited to MMPI-12, PUP-1 and tigapotide triflutate); agents that reduce the activity of epidermal growth factor receptor (EGFR) including but not limed to erlotinib, gefitinib, BMS-690514, cetuximab , antibodies targeting EGF receptor, inhibitors of EGF receptor kinase, and modulators of post-receptor signaling pathways; agents that reduce the activity of platelet derived growth factor (PDGF) (including but not limited to Imatinib mesylate (Novartis)) and also including PDGF neutralizing antibodies, antibodies targeting PDGF receptor (PDGFR), inhibitors of PDGFR kinase activity, and post-receptor signaling pathways; agents that reduce the activity of vascular endothelial growth factor (VEGF) (including but not limited to axitinib, bevacizumab, BIBF-1120, CDP-791, CT-322, IMC-18F1, PTC-299, and ramucirumab) and also including VEGF-neutralizing antibodies, antibodies targeting the VEGF receptor 1 (VEGFR1, Flt-1) and VEGF receptor 2 (VEGFR2, KDR), the soluble form of VEGFR1 (sFlt) and derivatives thereof which neutralize VEGF, and inhibitors of VEGF receptor kinase activity; inhibitors of multiple receptor kinases such as BIBF-1120 which inhibits receptor kinases for vascular endothelial growth factor, fibroblast growth factor, and platelet derived growth factor; agents that interfere with integrin function (including but not limited to STX-100 and IMGN-388) and also including integrin targeted antibodies; agents that interfere with the pro-fibrotic activities of IL-4 (including but not limited to AER-001, AMG-317, APG-201, and sIL-4Rα) and IL-13 (including but not limited to AER-001, AMG-317, anrukinzumab, CAT-354, cintredekin besudotox, MK-6105, QAX-576, SB-313, SL-102, and TNX-650) and also including neutralizing anti-bodies to either cytokine, antibodies that target IL-4 receptor or IL-13 receptor, the soluble form of IL-4 receptor or derivatives thereof that is reported to bind and neutralize both IL-4 and IL-13, chimeric proteins including all or part of IL-13 and a toxin particularly pseudomonas endotoxin, signaling though the JAK-STAT kinase pathway; agents that interfere with epithelial mesenchymal transition including inhibitors of mTor (including but not limited to AP-23573 or rapamycin); agents that reduce levels of copper such as tetrathiomolybdate; agents that reduce oxidative stress including N-acetyl cysteine and tetrathiomolybdate; and interferon gamma. Also contemplated are agents that are inhibitors of phosphodiesterase 4 (PDE4) (including but not limited to Roflumilast); inhibitors of phosphodiesterase 5 (PDE5) (including but not limited to mirodenafil, PF-4480682, sildenafil citrate, SLx-2101, tadalafil, udenafil, UK-369003, vardenafil, and zaprinast); or modifiers of the arachidonic acid pathway including cyclooxygenase and 5-lipoxegenase inhibitors (including but not limited to Zileuton), compounds that reduce tissue remodeling or fibrosis including prolyl hydrolase inhibitors (including but not limited to 1016548, CG-0089, FG-2216, FG-4497, FG-5615, FG-6513, fibrostatin A (Takeda), lufironil,P-1894B, and safironil) and peroxisome proliferator-activated receptor (PPAR)-gamma agonists (including but not limited to pioglitazone and rosiglitazone), and combinations thereof.
10A. A method of treating pulmonary fibrosis, optionally IPF, comprising (a) selecting a patient that exhibits (i) percent of predicted forced vital capacity volume (% FVC) of about 90% or less, or (ii) ratio of forced expiratory volume in one second (FEV1) to forced vital capacity volume (FVC) of about 0.80 or greater, or both, and (b) administering a therapeutically effective amount of an agent,
wherein the agent is selected from the group of BG-12, chemokine activity modulators (including but not limited to CNTO 888, an antibody targeting CCL2), Lys1 oxidase inhibitors (including but not limited to AB0024/GS-6624, an antibody targeting human lysyl oxidase-like 2), NOX4 inhibitors (including but not limited to GKT137831, a selective NOX 1/4 inhibitor), angiotensis II receptor antagonists (including but not limited to lorsartan), inhibitors or Wnt-beta catenin signaling agents (including but not limited to ICG-001); JNK inhibitors (including but not limited to CC930); IL-4/IL-13 antibody/soluble receptors (including but not limited to SAR156597), an LPA1/LPA3 antagonist (including but not limited to SAR-100842); a PG D₂ antagonist, a pirfenidone analog, and a deuterated pirfenidone (as described e.g., in W009/035598 and having one to fourteen deuterium atoms replacing a hydrogen atom in pirfenidone), nitric oxide (e.g., inhaled nitric oxide), a vitamin E and pentoxifylline combination (e.g., PTL-202 from Pacific Therapeutics), PXS25, desatinib (a multiple kinase inhibitor), PI3K/mTor dual inhibitor (e.g., BAY806946, XL765, GDC0980, GSK2126458, BEZ235, BGT226, PF04691502, PK1587, and/or SF1126), PI3K inhibitor (e.g., XL147, GDC0941, BKM120, PX866, ZSTK474, BYL719 (PI3K alpha), AMG319 (PI3K delta), CAL101 (PI3K delta), and/or GDC0032), 5-HT2A/B receptor antagonists (e.g., terguride), telomerase activator (e.g., TAT153), modulators (e.g., reducers) of chemokine activity (e.g., CNTO 888, an antibody that targets CCL2), Lys1 oxidase inhibitors (e.g., AB0024/GS-6624, a humanized mAb targeting human lysy1 oxidase-like 2), NOX4 inhibitor (e.g., GKT137831, a selective Nox 1/4 inhibitor), angiotensin II receptor antagonist (e.g., lorsartan), an anti aᵥI3₆ integrin agent, pentraxin (e.g., serum amyloid P, PTX-2, or PRM-151) and combinations thereof.
10B. A method of treating pulmonary fibrosis, optionally IPF, comprising (a) selecting a patient that exhibits (i) percent of predicted forced vital capacity volume (% FVC) of about 90% or less, or (ii) ratio of forced expiratory volume in one second (FEV1) to forced vital capacity volume (FVC) of about 0.80 or greater, or both, and (b) administering a therapeutically effective amount of an agent,
wherein the agent is an LPA1 receptor antagonist as disclosed herein.
11. The method of example 10 or 10A or 10 B, further comprising administering pirfenidone.
12. The method of example 11, wherein the pirfenidone comprises a deuterated pirfenidone as described herein.

1. A method of treating pulmonary fibrosis, comprising
(a) selecting a patient with pulmonary fibrosis that exhibits percent of predicted forced vital capacity volume (% FVC) of about 80% or less
(b) administering a therapeutically effective amount of pirfenidone.

2. The method of claim 1, wherein the patient exhibits ratio of forced expiratory volume in one second (FEV1) to forced vital capacity volume (FVC) of about 0.80 or greater, or both.

3. (canceled)

4. A method of treating pulmonary fibrosis, comprising
(a) selecting a patient with pulmonary fibrosis that exhibits (i) percent of predicted forced vital capacity volume (% FVC) of about 80% or less, or (ii) ratio of forced expiratory volume in one second (FEV1) to forced vital capacity volume (FVC) of about 0.80 or greater, or both, and
(b) administering a therapeutically effective amount of an agent, wherein the agent is a steroid, a cytotoxic agent, bardoxolone, a LPA agonist; Torisel (temsirolimus); a PI3K inhibitor; pentraxin or serum amyloid P; a MEK inhibitor; a p38 inhibitor; a PAI-1 inhibitor; an agent that reduces the activity of transforming growth factor-beta (TGF-β); lerdelimumab; metelimumab; an antibody that targets one or more TGF-β isoforms, an inhibitor of TGF-β receptor kinases TGFBR1 and TGFBR2; a modulator of post-receptor signaling pathways; a modulator of chemokine receptor signaling; an endothelin receptor antagonist; an agent that reduces the activity of connective tissue growth factor (CTGF); a matrix metalloproteinase (MMP) inhibitor; an agent that reduces the activity of epidermal growth factor receptor (EGFR); an antibody that targets EGF receptor; an inhibitor of EGF receptor kinase, a modulator of post-receptor signaling pathways; an agent that reduces the activity of platelet derived growth factor (PDGF); a PDGF neutralizing antibody; an antibody targeting PDGF receptor (PDGFR); an inhibitor of PDGFR kinase activity; an inhibitor of post-receptor signaling pathways; an agent that reduces the activity of vascular endothelial growth factor (VEGF); a VEGF-neutralizing antibody; an antibody targeting the VEGF receptor 1 (VEGFR1, Flt-1) and VEGF receptor 2 (VEGFR2, KDR); an inhibitor of VEGF receptor kinase activity; an inhibitor of multiple receptor kinases; an agent that interferes with integrin function; an integrin targeted antibody; an agent that interferes with the pro-fibrotic activity of IL-4, an agent that interferes with the pro-fibrotic activity of IL-13; a neutralizing antibody to IL-4 or IL-13, an antibody that targets IL-4 receptor or IL-13 receptor, or both; a chimeric protein including all or part of IL-13 and a toxin particularly pseudomonas endotoxin; an agent that interferes with epithelial mesenchymal transition; an inhibitor of mTor; an agent that reduces levels of copper; an agent that reduces oxidative stress; an agent that reduces interferon gamma; a PDE4 inhibitor; a PDE5 inhibitor; a modifier of the arachidonic acid pathway; a cyclooxygenase inhibitor; a 5-lipoxegenase inhibitor; an agent that reduces tissue remodeling or fibrosis; a prolyl hydrolase inhibitor; a peroxisome proliferator-activated receptor (PPAR)-gamma agonist, or a combination thereof.

5. The method of claim 4, where in the agent is prednisolone, azathioprine, cyclophosphamide, bardoxolone, AM152, temsirolimus, PTX-2, PRM-151; ARRY-162; ARRY-300; Tiplaxtinin; GC-1008; lerdelimumab; CAT-152; Trabio; metelimumab; CAT-192; LY-2157299; ACU-HTR-028 ; ALKS; ambrisentan; avosentan; bosentan; clazosentan; darusentan; BQ-153; FR-139317, L-744453; macitentan; PD-145065; PD-156252; PD163610;PS-433540; S-0139; sitaxentan sodium; TBC-3711; zibotentan; FG-3019; MMPI-12, PUP-1; tigapotide triflutate; erlotinib, gefitinib, BMS-690514, cetuximab; Imatinib mesylate; axitinib, bevacizumab, BIBF-1120, CDP-791, CT-322, IMC-18F1, PTC-299, ramucirumab; BIBF-1120; STX-100; IMGN-388; AER-001, AMG-317, APG-201, sIL-4Ra; anrukinzumab, CAT-354, cintredekin besudotox, MK-6105, QAX-576, SB-313, SL-102; AP-23573; rapamycin; tetrathiomolybdate; N-acetyl cysteine; Roflumilast; mirodenafil, PF-4480682, sildenafil citrate, SLx-2101, tadalafil, udenafil, UK-369003, vardenafil, zaprinast; Zileuton, 1016548, CG-0089, FG-2216, FG-4497, FG-5615, FG-6513, fibrostatin A, lufironil,P-1894B, safironil; pioglitazone; rosiglitazone, or a combination thereof.

6-8. (canceled)

9. The method of claim 2, wherein % FVC ranges from about 50% to about 80%.

10. The method of claim 2, wherein the patient has been diagnosed with pulmonary fibrosis, optionally IPF, for at least six months, and optionally less than 48 months.

11. The method of claim 2, wherein the patient exhibits a diffusion capacity (% DLco) ranging from about 30% to about 90%.

12. The method of claim 2, wherein the pirfenidone is administered at a total daily dosage of at least about 1800 mg.

13. The method of claim 2, wherein the pirfenidone is administered at a total daily dosage of about 2403 mg.

14. The method of claim 13, wherein the pirfenidone is administered to the patient three times per day, with food.

15. The method of claim 2, wherein the pirfenidone comprises a deuterated pirfenidone.

16. The method of claim 4 further comprising administering pirfenidone.

17. (canceled)

18. The method of claim 2, wherein the patient has idiopathic pulmonary fibrosis (IPF).

19. The method of claim 4, wherein % FVC ranges from about 50% to about 80%.

20. The method of claim 4 wherein the patient has been diagnosed with pulmonary fibrosis, for at least six months, and optionally less than 48 months.

21. The method of claim 4, wherein the patient exhibits a diffusion capacity (% DLco) ranging from about 30% to about 90%.

22. The method of claim 4, wherein the patient has idiopathic pulmonary fibrosis (IPF).
